(19) United States
(12) Patent Application Publication
(10) Pub. No.: US 2024/0180908 A1
(43) Pub. Date: Jun. 6, 2024

(54) TREATMENT OF KIDNEY DISEASES

(71) Applicant: ACLIPSE ONE, INC., Radnor, PA (US)

(72) Inventors: Ning Shan, Chandler, AZ (US); Ira N. Kalfus, New York, NY (US)

(21) Appl. No.: 18/553,533

(22) PCT Filed: Mar. 30, 2022
(86) PCT No.: PCT/US2022/022573

§ 371 Date: Sep. 29, 2023

Related U.S. Application Data
(60) Provisional application No. 63/169,678, filed on Apr. 1, 2021.

(51) Int. Cl. A61K 31/506; A61K 31/4439; A61P 13/12

(52) U.S. Cl. CPC A61K 31/506; A61K 31/4439; A61P 13/12

(57) ABSTRACT

Disclosed herein are thiazolidinediones for the treatment of kidney diseases, glomerular diseases, and nephrotic syndrome.

### CROSS-REFERENCE TO RELATED APPLICATIONS

The present application claims the benefit of U.S. Provisional Patent Application 63/169,678 filed Apr. 1, 2021, the entire contents of which are incorporated by reference herein.

### FIELD

The present disclosure relates to a therapeutic agent and methods for the treatment of glomerular diseases and nephrotic syndrome.

### BACKGROUND

Many diseases affect kidney function by attacking the glomeruli, the tiny units within the kidney where blood is cleaned. Glomerular diseases include many conditions with a variety of genetic and environmental causes, but they fall into two major categories: (1) glomerulonephritis comprising inflammation of the membrane tissue in the kidney that serves as a filter, separating wastes and extra fluid from the blood, and (2) glomerulosclerosis comprising the scarring or hardening of the tiny blood vessels within the kidney. Although glomerulonephritis and glomerulosclerosis have different causes, they can both lead to kidney failure.

Patients with glomerular diseases have significant amounts of protein in the urine, which may be referred to as “nephrotic range” if levels are very high. Red blood cells in the urine are a frequent finding as well, particularly in some forms of glomerular disease.

Glomerular disease can be caused by a variety of factors. It may be the direct result of an infection or a drug toxic to the kidneys, or it may result from a disease that affects the entire body, like diabetes or lupus. Many different kinds of diseases can cause swelling or scarring of the nephron or glomerulus. Sometimes glomerular disease is idiopathic. Glomerular disease can be as a result of an autoimmune diseases, such as systemic lupus erythematosus (SLE), anti-GBM (Goodpasture syndrome), or immunoglobulin A (IgA) nephropathy; hereditary nephritis or Alport syndrome; infection-related glomerular disease, such as acute post-streptococcal glomerulonephritis (PSGN), bacterial endocarditis, and human immunodeficiency disease {HIV); sclerotic diseases, such as diabetic nephropathy and focal segmental glomerulosclerosis (FSGS); other glomerular diseases, such as membranous nephropathy and minimal change diseases (MCD); and chronic kidney disease.

Kidney failure is the acute or chronic loss of 85 percent or more kidney function. End-stage renal disease (ESRD) is kidney failure that is treated by dialysis or kidney transplant. Depending on the form of glomerular disease, kidney function may be lost in a matter of days or weeks or may deteriorate slowly and gradually over the course of decades.

Acute renal failure includes a few forms of glomerular disease cause very rapid deterioration of kidney function. For example, PSGN can cause severe symptoms (hematuria, proteinuria, edema) within 2 to 3 weeks after a sore throat or skin infection develops. The patient may temporarily require dialysis to replace renal function. This rapid loss of kidney function is called acute renal failure (ARF). Although ARF can be life-threatening while it lasts, kidney function usually returns after the cause of the kidney failure has been treated. In many patients, ARF is not associated with any permanent damage. However, some patients may recover from ARF and subsequently develop chronic kidney disease.

Nephrotic syndrome is one of the most common kidney diseases seen in children and adults. It is a remitting and relapsing disease characterized by massive loss of serum proteins into the urine through a damaged glomerular filtration barrier, leading to hypoalbuminemia and swelling throughout the body (edema). Podocytes are a key component of the kidney's filtration barrier, and during nephrotic syndrome, they undergo dramatic structural alterations in the foot processes that attach these cells to the glomerular basement membrane.

For the last 50 years the primary therapy for nephrotic syndrome has been oral glucocorticoids. Unfortunately, glucocorticoids have serious side effects, and in approximately 20% of patients, they are ineffective in inducing clinical remission of disease (i.e., steroid-resistant nephrotic syndrome). Thus, alternative therapies with greater efficacy and/or less severe side effects are critically needed. Thiazolidinediones represents a novel therapeutic strategy that could slow, halt, or reverse the underlying disease process in diseases involving glomerular diseases, kidney failures and end-stage renal diseases, such as acute renal failure, kidney failure and nephrotic syndrome.

### SUMMARY

Disclosed herein are methods of protecting kidney cells from damage and improve cell viability, comprising a step of contacting the cell with an effective amount of a thiazolidinedione selected from one or more of rosiglitazone, lobeglitazone, ciglitazone, darglitazone, englitazone, netoglitazone, rivoglitazone, troglitazone, and balaglitazone. In some embodiments, the thiazolidinedione is lobeglitazone. In some embodiments, the thiazolidinedione is not rosiglitazone.

In some embodiments, the kidney cell is from a kidney tissue selected from renal cortex, renal medulla, renal papilla, renal pyramids, renal columns, fibrous capsule, hilum, renal artery, renal vein, renal pelvis, ureter, major calyx, and minor calyx. In some embodiments, the kidney cell is a kidney glomerulus parietal cell, a kidney glomerulus podocyte, a kidney proximal tubule brush border cell, a loop of Henle thin segment cell, a thick ascending limb cell, a kidney distal tubule cell, a collecting duct principal cell, a collecting duct intercalated cell, and an interstitial kidney cell. In some embodiments, the cell is an animal cell. In some embodiments, the cell in a human cell. In some embodiments, the cell is treated in vitro. In some embodiments, the cell is treated ex vivo. In some embodiments, the cell is treated in vivo.

In some embodiments, the cell is in a subject having a disease or disorder or is at risk of the disease or disorder. In some embodiments, the cell is from an animal having a disease or disorder or at risk of acquiring the disease or disorder selected from any one or more of age-associated glomerulonephropathy, AL amyloidosis, Alport syndrome, amyloidosis (amyloid nephropathy), ANCA vasculitis, anti-GBM disease (Goodpasture syndrome), C1q nephropathy, C3 glomerulopathy, collapsing glomerulopathy, collapsing glomerulonephropathy, congenital nephrotic syndrome of the Finnish type (CNSF), cryoglobulinemia, diabetes, Denys-Drash Syndrome, diabetic glomerulonephropathy, diabetic nephropathy, diffuse mesangial sclerosis (DMS), Fabry disease, fibrillary glomerulonephritis (GN), focal segmental glomerulosclerosis (FSGS), heavy chain deposition disease, hypertensive nephropathy, IgA vasculitis, IgA nephropathy, IgM nephropathy, immune and inflammatory glomerulonephropathy, immunotactoid glomerulopathy, light chain deposition disease, lupus, lupus nephritis, membranous nephropathy, membranoproliferative glomerulonephritis (MPGN), mesangial proliferation, mesangial sclerosis, myeloma kidney, minimal change disease, nephrotic syndrome, post-infectious glomerulonephritis (GN), thin basement membrane (TBM), thrombotic microangiopathy (TMA), or a condition associated therewith. In some embodiments, the nephrotic syndrome is frequent relapsing nephrotic syndrome, steroid dependent nephrotic syndrome, or steroid resistant nephrotic syndrome.

Also disclosed herein are methods of treating a disease or disorder in a subject in need thereof, the method comprising administering to the subject a therapeutically effective amount of a pharmaceutical composition comprising a thiazolidinedione selected from one or more of rosiglitazone, lobeglitazone, ciglitazone, darglitazone, englitazone, netoglitazone, rivoglitazone, troglitazone, and balaglitazone. In some embodiments, the thiazolidinedione is lobeglitazone. In some embodiments, the thiazolidinedione is not rosiglitazone.

In some embodiments, the disease or disorder is selected from any one or more of age-associated glomerulonephropathy, AL amyloidosis, Alport syndrome, amyloidosis (amyloid nephropathy), ANCA vasculitis, anti-GBM disease, C1q nephropathy, C3 glomerulopathy, collapsing glomerulopathy, collapsing glomerulonephropathy, CNSF, cryoglobulinemia, diabetes, Denys-Drash Syndrome, diabetic glomerulonephropathy, diabetic nephropathy, DMS, Fabry disease, fibrillary GN, FSGS, heavy chain deposition disease, hypertensive nephropathy, IgA vasculitis, IgA nephropathy, IgM nephropathy, immune and inflammatory glomerulonephropathy, immunotactoid glomerulopathy, light chain deposition disease, lupus, lupus nephritis, membranous nephropathy, MPGN, mesangial proliferation, mesangial sclerosis, myeloma kidney, minimal change disease, nephrotic syndrome, post-infectious GN, TBM, TMA, or a condition associated therewith. In some embodiments, the nephrotic syndrome is frequent relapsing nephrotic syndrome, steroid dependent nephrotic syndrome, or steroid resistant nephrotic syndrome. In some embodiments, the disease or disorder is selected from any one or more of chronic kidney disease, nephrotic syndrome, glomerular disease, focal segmental glomerulosclerosis (FSGS), and mesangial sclerosis. In some embodiments, the subject is a mammal. In some embodiments, the mammal is a non-human animal. In some embodiments, the mammal is a human. In some embodiments, the thiazolidinedione is administered at a dose of 0.01 mg or higher. In some embodiments, the thiazolidinedione is administered at a dose between 0.01-5000 mg/day.

Also disclosed herein is the use of a thiazolidinedione selected from one or more of rosiglitazone, lobeglitazone, ciglitazone, darglitazone, englitazone, netoglitazone, rivoglitazone, troglitazone, and balaglitazone to reduce the levels of protein excreted in the urine. In some embodiments, the level of protein excreted in the urine is measured as frequency at which protein excretion occurs. In some embodiments, the level of protein excreted in the urine is measured as rate at which protein excretion occurs.

Also disclosed herein is the use of a thiazolidinedione selected from one or more of rosiglitazone, lobeglitazone, ciglitazone, darglitazone, englitazone, netoglitazone, rivoglitazone, troglitazone, and balaglitazone, to reduce the use of glucocorticoids in the treatment of a kidney diseases such as nephrotic syndrome or glomerular disease. In some embodiments, the nephrotic syndrome is FRNS or SDNS.

Also disclosed herein is the use of a thiazolidinedione selected from one or more of rosiglitazone, lobeglitazone, ciglitazone, darglitazone, englitazone, netoglitazone, rivoglitazone, troglitazone, and balaglitazone, to reduce the risk for glucocorticoid-induced toxicity in a subject with a kidney disease.

Also disclosed here is the use of a thiazolidinedione selected from one or more of rosiglitazone, lobeglitazone, ciglitazone, darglitazone, englitazone, netoglitazone, rivoglitazone, troglitazone, and balaglitazone, to slow down, stop, or reverse disease progress of kidney disease.

In some embodiments, the thiazolidinedione is lobeglitazone. In some embodiments, the thiazolidinedione is not rosiglitazone.

Also disclosed herein are methods of treating nephrotic syndrome in a mammal comprising administering an effective amount of a thiazolidinedione selected from one or more of rosiglitazone, lobeglitazone, ciglitazone, darglitazone, englitazone, netoglitazone, rivoglitazone, troglitazone, and balaglitazone to the mammal. In some embodiments, the nephrotic syndrome is FRNS or SDNS.

Also disclosed herein are methods of treating glomerular disease in a mammal comprising administering an effective amount of a thiazolidinedione selected from one or more of rosiglitazone, lobeglitazone, ciglitazone, darglitazone, englitazone, netoglitazone, rivoglitazone, troglitazone, and balaglitazone to the mammal. In some embodiments, the glomerular disease is focal segmental glomerulosclerosis (FSGS), minimal change disease, or membranous nephropathy.

In some embodiments, the thiazolidinedione is lobeglitazone. In some embodiments, the thiazolidinedione is not rosiglitazone.

### BRIEF DESCRIPTION OF THE DRAWINGS

FIG. 1 depicts the effects of the disclosed compounds on the proliferation of kidney cells (CIHP-1) pre-treated with puromycin aminonucleoside (PAN) for three days.

FIG. 2 depicts the effects of the disclosed compounds on the proliferation of CIHP-1 cells pre-treated with PAN for five days.

### DETAILED DESCRIPTION

Disclosed herein are methods of treating a disease or disorder disclosed herein with a thiazolidinedione, wherein the disease or disorder involves the kidneys as disclosed herein.

The term “thiazolidinedione” refers to a class of heterocyclic glitazones compounds which comprise a five-membered C3NS ring, including prodrugs, salts, solvates, hydrates, cocrystals, enantiomers, and deuterated forms thereof. As used herein, a thiazolidinedione includes, but is not limited to, one or more of pioglitazone, rosiglitazone, lobeglitazone, ciglitazone, darglitazone, englitazone, netoglitazone, rivoglitazone, troglitazone, balaglitazone, and other thiazolidinedione molecules. In some embodiments disclosed herein, the methods include using any thiazolidinedione. In some embodiments, the thiazolidinedione is lobeglitazone. In some embodiments, the methods specifically exclude use of one or more thiazolidinedione disclosed herein. In some embodiments, the thiazolidinedione is not pioglitazone. In some embodiments, the thiazolidinedione is not rosiglitazone.

Lobeglitazone (Duvie®, Chong Kun Dang) is a thiazolidinedione with a chemical name of 5-(4-(2-((6-(4-Methoxyphenoxy)pyrimidin-4-yl)(methyl)amino)ethoxy)benzyl) thiazolidine-2,4-dione. As an agonist for both PPARα and PPARγ, lobeglitazone works as an insulin sensitizer by binding to the PPAR receptors in fat cells and making the cells more responsive to insulin.

As used herein, the terms “treat”, “treating”, or “treatment” means to alleviate, reduce or abrogate one or more symptoms or characteristics of a disease and may be curative, palliative, prophylactic or slow the progression of the disease.

The term “effective amount” means an amount that will result in reduction of, as applicable or specified, damage to kidney, and in a desired effect or result. The term ‘therapeutically effective amount’ means an amount of a thiazolidinedione comprising, but not limited to, one or more of pioglitazone, rosiglitazone, lobeglitazone, ciglitazone, darglitazone, englitazone, netoglitazone, rivoglitazone, troglitazone, balaglitazone, and other thiazolidinedione molecules, alone or combined with other active ingredients, that will elicit a desired biological or pharmacological response, e.g., effective to prevent, alleviate, or ameliorate symptoms of a disease or disorder; slow, halt or reverse an underlying disease process or progression; partially or fully restore cellular function; or prolong the survival of the subject being treated. In some embodiments, the thiazolidinedione is not pioglitazone. In some embodiments, the thiazolidinedione is not rosiglitazone.

The term “patient” or “subject” includes mammals, including non-human animals and especially humans. In one embodiment the patient or subject is a human. In another embodiment, the patient or subject is a human male. In another embodiment, the patient or subject is a human female. In another embodiment, the patient or subject is of any age.

The term “significant” or “significantly” is determined by t-test at 0.05 level of significance.

The present methods are predicted on the surprising finding that thiazolidinediones can significantly protect podocytes against puromycin aminonucleoside (PAN)-induced injury (designed to mimic nephrotic syndrome-related injury), as determined by both cell survival and actin cytoskeletal integrity. Therefore, disclosed herein is the use of one or more thiazolidinedione for the treatment of kidney diseases, glomerular diseases, and nephrotic syndrome (including frequent relapsing nephrotic syndrome [FRNS], steroid dependent nephrotic syndrome [SDNS], or steroid resistant nephrotic syndrome).

Kidney diseases that are driven by damage that reduce function of the kidney, and can be treated by a thiazolidinedione disclosed herein include chronic kidney disease, kidney stones, glomerular diseases or glomerulonephritis, polycystic kidney disease, urinary tract infections, or a condition associated therewith.

Glomerular diseases that are driven by injury to the glomeruli include, but are not limited to, age-associated glomerulonephropathy, AL amyloidosis, Alport syndrome, amyloidosis (amyloid nephropathy), ANCA (anti-neutrophilic cytoplasmic antibody) vasculitis, anti-GBM disease (Goodpasture syndrome), C1q nephropathy, C3 glomerulopathy, collapsing glomerulopathy, collapsing glomerulonephropathy, congenital nephrotic syndrome of the Finnish type (CNSF), cryoglobulinemia, diabetes, Denys-Drash Syndrome, diabetic glomerulonephropathy, diabetic nephropathy, diffuse mesangial sclerosis (DMS), Fabry disease (Anderson-Fabry disease), fibrillary glomerulonephritis (GN), focal segmental glomerulosclerosis (FSGS), heavy chain deposition disease, hypertensive nephropathy, IgA vasculitis (formerly Henoch-Schönlein Purpura or HSP), IgA nephropathy, IgM nephropathy, immune and inflammatory glomerulonephropathy, immunotactoid glomerulopathy, light chain deposition disease, lupus, lupus nephritis, membranous nephropathy, membranoproliferative glomerulonephritis (MPGN), mesangial proliferation, mesangial sclerosis, myeloma kidney, minimal change disease, nephrotic syndrome (including but not limited to frequent relapsing nephrotic syndrome [FRNS], steroid dependent nephrotic syndrome [SDNS], and steroid resistant nephrotic syndrome), post-infectious glomerulonephritis (GN), thin basement membrane (TBM), thrombotic microangiopathy (TMA), or a condition associated therewith.

In some aspects, the present disclosure provides a method of exerting protective effects in a cell, comprising contacting the cell with an effective amount of a thiazolidinedione As used herein, the term “effective amount” refers to an amount of thiazolidinedione that will result in the desired effect or result, e.g., an amount that will result in protective effect.

In some aspects, the disclosure provides a method of increasing cell lifespan, comprising the step of contacting the cell with an effective amount of a thiazolidinedione.

In some embodiments, a cell referred to herein is a kidney cell from a kidney tissue selected from renal cortex, renal medulla, renal papilla, renal pyramids, renal columns, fibrous capsule, hilum, renal artery, renal vein, renal pelvis, ureter, major calyx, and minor calyx. In some embodiments, the kidney cell is selected from a kidney glomerulus parietal cell, a kidney glomerulus podocyte, a kidney proximal tubule brush border cell, a loop of Henle thin segment cell, a thick ascending limb cell, a kidney distal tubule cell, a collecting duct principal cell, a collecting duct intercalated cell, and an interstitial kidney cell.

In some embodiments, the cell is an animal cell, e.g., a mammalian cell. In some embodiments, the cell in a human cell or non-human cell. In some embodiments, the cell is treated in vitro, in vivo, or ex vivo. In some embodiments, the cell is a diseased cell. In some embodiments, the cell is diseased cell from a patient suffering from a disease or disorder disclosed herein.

Also disclosed herein are methods of treating an animal having a disease or disorder that would benefit from the protective effect on cells, or for preventing or reducing the risk of acquiring a disease or disorder in an animal, the method comprising the step of administering a therapeutically effective amount of a pharmaceutical composition comprising a thiazolidinedione to the animal. In some embodiments, the animal is a mammal. In some embodiments, the mammal is a human or a non-human mammal. In some embodiments, the mammal is a human. In some embodiments, the disease or disorder is caused by damage that reduces function of the kidney. In some embodiments, the disease is selected from one or more of chronic kidney disease, kidney stones, glomerular diseases or glomerulonephritis, polycystic kidney disease, urinary tract infections, or a condition associated therewith. In some embodiments, the disease is a glomerular disease that driven by injury of glomeruli.

Also disclosed herein are methods for treating a disease or disorder resulting in accelerated damage of kidney in an animal or increasing lifespan of a cell in a patient with a disease or disorder resulting in damage to the kidney. In some embodiments, the method comprising the step of administering a therapeutically effective amount of a pharmaceutical composition comprising a thiazolidinedione to the animal. In some embodiments, the animal is a mammal. In some embodiments, the mammal is a human or a non-human mammal. In one embodiment, the disease or disorder is selected from, but not limited to, chronic kidney disease, kidney stones, glomerular diseases or glomerulonephritis, polycystic kidney disease, urinary tract infections, age-associated glomerulonephropathy, AL amyloidosis, Alport syndrome, amyloidosis (amyloid nephropathy), ANCA vasculitis, anti-GBM disease (Goodpasture syndrome), C1q nephropathy, C3 glomerulopathy, collapsing glomerulopathy, collapsing glomerulonephropathy, congenital nephrotic syndrome of the Finnish type (CNSF), cryoglobulinemia, diabetes, Denys-Drash Syndrome, diabetic glomerulonephropathy, diabetic nephropathy, diffuse mesangial sclerosis (DMS), Fabry disease (Anderson-Fabry disease), fibrillary glomerulonephritis (GN), focal segmental glomerulosclerosis (FSGS), heavy chain deposition disease, hypertensive nephropathy, IgA vasculitis (formerly Henoch-Schönlein Purpura or HSP), IgA nephropathy, IgM nephropathy, immune and inflammatory glomerulonephropathy, immunotactoid glomerulopathy, light chain deposition disease, lupus, lupus nephritis, membranous nephropathy, membranoproliferative glomerulonephritis (MPGN), mesangial proliferation, mesangial sclerosis, myeloma kidney, minimal change disease, nephrotic syndrome (including but not limited to frequent relapsing nephrotic syndrome [FRNS], steroid dependent nephrotic syndrome [SDNS], and steroid resistant nephrotic syndrome), post-infectious glomerulonephritis (GN), thin basement membrane (TBM), thrombotic microangiopathy (TMA), or a condition associated therewith.

Also disclosed herein are methods of treating nephrotic syndrome. In some embodiments, the nephrotic syndrome includes primary nephrotic syndrome and secondary nephrotic syndrome. In some embodiments, the primary nephrotic syndrome is idiopathic. In some embodiments, the secondary nephrotic syndrome is caused by a disease such as diabetes, cancer, or infections, or a drug side effect. In some embodiments, the nephrotic syndrome includes frequent relapsing nephrotic syndrome (FRNS), steroid dependent nephrotic syndrome (SDNS), or steroid resistant nephrotic syndrome.

In another aspect, provided herein are methods of reducing protein excreted in the urine, in terms of frequency or rate at which protein excretion occurs; reducing the use of glucocorticoids for treatment of kidney diseases such as nephrotic syndrome and glomerular disease; reducing the risk for glucocorticoids-induced toxicity in the treatment of kidney disease; or slowing, stopping, or reversing disease progression to chronic kidney disease.

In another aspect, disclosed herein are methods of treating an animal having a disease or disorder with a symptom that is prevented, alleviated, or ameliorated by cell protection; or with a disease process or progression that slowed, halted or reversed by cell protection; the method comprising administering a therapeutically effective amount of a pharmaceutical composition comprising a thiazolidinedione. In some embodiments, the thiazolidinedione is lobeglitazone.

In a related aspect, disclosed herein are methods of treating nephrotic syndrome. In one embodiment, the nephrotic syndrome includes primary nephrotic syndrome and secondary nephrotic syndrome.

The present disclosure further provides of the use of a thiazolidinedione for the preparation of a medicament for treating a human having any one of the diseases or disorders disclosed herein or for use in any method of the present disclosure involving the administration of a thiazolidinedione to a human.

In another aspect, the present disclosure provides for an in vitro method of screening a candidate therapeutic agent(s) for its ability to cell protection, the method comprising the steps of (a) exposing PAN-treated podocytes to the candidate therapeutic; (b) comparing the number of survived cells between podocytes exposed to the candidate therapeutic and control cells, e.g., PAN-treated podocytes that are not exposed to the candidate therapeutic (i.e., unexposed PAN-treated podocytes).

The pharmaceutical compositions of the present disclosure comprise a therapeutically effective amount of a thiazolidinedione and at least one pharmaceutically acceptable excipient. The term “excipient” refers to a pharmaceutically acceptable, inactive substance used as a carrier for the pharmaceutically active ingredient thiazolidinedione, and includes antiadherents, binders, coatings, disintegrants, fillers, diluents, solvents, flavors, bulkants, colors, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

A pharmaceutical formulation of the present disclosure may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of thiazolidinedione and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of thiazolidinedione administered to a patient in a single dose.

In some embodiments, a pharmaceutical composition disclosed herein is delivered to a subject via a parenteral route, an enteral route, or a topical route.

Examples of parental routes suitable for use with the disclosed pharmaceutical compositions include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous. In some embodiments, the route of administration is not buccal or sublingual administration. In some embodiments, the route of administration is not nasal administration.

Enteral routes of administration include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories.

Topical administration includes administration to a body surface, such as skin or mucous membranes, including pulmonary administration. Transdermal forms include cream, foam, gel, lotion or ointment. Pulmonary forms include liquids and powders, e.g., liquid spray.

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of thiazolidinedione administered to a patient is selected from up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, 2 mg, 1 mg, 0.9 mg, 0.8 mg, 0.7 mg, 0.6 mg, 0.5 mg, 0.45 mg, 0.4 mg, 0.3 mg, 0.2 mg, 0.1 mg, 0.08 mg, 0.05 mg, 0.03 mg, 0.02 mg or up to 0.01 mg. In another embodiment, the daily dose is at least 0.01 mg, 0.02 mg, 0.05 mg, 0.08 mg, 0.1 mg, 0.2 mg, 0.3 mg, 0.4 mg, 0.45 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1,000 mg, 2,000 mg, 3,000 mg, 4,000 mg, or at least 5,000 mg. In another embodiment, the daily dose is 0.01-0.0.2 mg, 0.02-0.05 mg, 0.05-0.08 mg, 0.08-0.1 mg, 0.1-0.2 mg, 0.2-0.4 mg, 0.4-0.6 mg, 0.6-0.8 mg, 0.8-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, 5-200 mg, 5-300 mg, 5-400 mg, 5-500 mg, 5-600 mg, 5-700 mg, 5-800 mg, 5-900 mg, 5-1,000 mg, 5-2,000 mg, 5-5,000 mg or more than 5,000 mg, or any range bound by a pair of these values.

In another embodiment, a single dose of thiazolidinedione administered to a patient is selected from: 0.01 mg, 0.02 mg, 0.05 mg, 0.08 mg, 0.1 mg, 0.2 mg, 0.3 mg, 0.4 mg, 0.45 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg 490 mg, 500 mg, 600 mg, 700 mg, 800 mg, 900 mg, 1,000 mg, 2,000 mg, 3,000 mg, 4,000 mg, or 5,000 mg, or any range bound by a pair of these values. In one embodiment, the single dose is administered by oral administration. In another embodiment, the single dose is administered by injection, e.g., subcutaneous, intramuscular, or intravenous. In another embodiment, the single dose is administered by inhalation administration. In some embodiments, the thiazolidinedione is lobeglitazone.

As a non-limited example, the dose of a thiazolidinedione, such as lobeglitazone, administered by oral administration may be about 0.01 to 50 mg per day to be administered in divided doses. A single dose of a thiazolidinedione, such as lobeglitazone, administered by subcutaneous injection may be about 0.01-50 mg, preferably about 0.1-10 mg, 0.2-1 mg, 0.3-0.6 mg or 0.5 mg, or any range bound by a pair of these values. Other embodiments include ranges of about 0.05-5,000 mg, preferably about 0.1-10 mg, 0.2-5 mg, 0.3-1 mg, or 0.5 mg, or any range bound by a pair of these values. Subcutaneous infusion may be preferable in those patients requiring division of injections into more than 10 doses daily.

The fine particle dose of a thiazolidinedione, such as lobeglitazone, administered by pulmonary administration, e.g., inhalation using a pressurized metered dose inhaler (pMDI), dry powder inhaler (DPI), soft-mist inhaler, nebulizer, or other device, may be in the range of about, 0.1-50 mg, preferably about 0.2-10 mg, 0.3-1 mg, or 0.5 mg, or any range bound by a pair of these values. Other embodiments include ranges of about 0.05-5,000 mg, preferably about 0.1-1,000 mg, 0.2-100 mg, 0.3-1 mg, 0.4-0.5 mg, or 0.5 mg, or any range bound by a pair of these values. The Nominal Dose (ND), i.e., the amount of drug metered in the receptacle (also known as the metered dose), of a thiazolidinedione, such as lobeglitazone, administered by pulmonary administration may be, for example, in the range of 0.1-15 mg, 0.1-10 mg, 0.1-1 mg, 0.2-0.3 mg, 0.3-0.4 mg, 0.4-0.5 mg, 0.5-0.6 mg, 0.6-0.7 mg, 0.7-0.8 mg, 0.8-0.9 mg, or 0.9-1 mg, or any range bound by a pair of these values. Other embodiments include ranges of about 0.05-5,000 mg, preferably about 0.1-1,000 mg, 0.2-10 mg, 0.3-1 mg, 0.4-0.5 mg, or 0.5 mg, or any range bound by a pair of these values.

Long-acting pharmaceutical compositions may be administered, 1, 2, 3, 4, 5, 6, 7, 8, 9, 10 or more than 10 times daily (preferably ≤10 times per day), every other day, every 3 to 4 days, every week, or once every two weeks depending on half-life and clearance rate of the particular formulation.

In an embodiment of any of the above methods and compositions, a thiazolidinedione, or its salt, solvates, hydrates, and co-crystals thereof, is a racemic mixture of R and S enantiomers, or enriched in R enantiomer (i.e., the ratio of R to S enantiomer being administered, is from 1.1:1 to 1,000:1, from 10:1 to 10,000:1, or from 100:1 to 100,000:1, or over all thiazolidinedione enantiomers in the composition is at least 98% R enantiomer, 99% enantiomer, 99.5% enantiomer, 99.9% enantiomer, or is free of any observable amount of S enantiomer), or enriched in S enantiomer (i.e., the ratio of S to R enantiomer is from 1.1:1 to 1,000:1, from 10:1 to 10,000:1, or from 100:1 to 100,000:1, or over all thiazolidinedione enantiomers in the composition is at least 98% S enantiomer, 99% enantiomer, 99.5% enantiomer, 99.9% enantiomer, or is free of any detectable amount of R enantiomer).

The present disclosure further provides an in vitro or ex vivo method of reducing cell damage, the method comprising the step of contacting the cell with an effective amount of a thiazolidinedione.

Suitably, the cell is having a disease or disorder or at risk of the disease or disorder or at risk of acquiring the disease or disorder selected from any one or more of: chronic kidney disease, kidney stones, glomerular diseases or glomerulonephritis, polycystic kidney disease, urinary tract infections, age-associated glomerulonephropathy, AL amyloidosis, Alport syndrome, amyloidosis (amyloid nephropathy), ANCA vasculitis, anti-GBM disease, C1q nephropathy, C3 glomerulopathy, collapsing glomerulopathy, collapsing glomerulonephropathy, congenital nephrotic syndrome of the Finnish type (CNSF), cryoglobulinemia, diabetes, Denys-Drash Syndrome, diabetic glomerulonephropathy, diabetic nephropathy, diffuse mesangial sclerosis (DMS), Fabry disease (Anderson-Fabry disease), fibrillary glomerulonephritis (GN), focal segmental glomerulosclerosis (FSGS), heavy chain deposition disease, hypertensive nephropathy, IgA vasculitis (formerly Henoch-Schonlein Purpura or HSP), IgA nephropathy, IgM nephropathy, immune and inflammatory glomerulonephropathy, immunotactoid glomerulopathy, light chain deposition disease, lupus, lupus nephritis, membranous nephropathy, membranoproliferative glomerulonephritis (MPGN), mesangial proliferation, mesangial sclerosis, myeloma kidney, minimal change disease, nephrotic syndrome (including but not limited to frequent relapsing nephrotic syndrome or FRNS, steroid dependent nephrotic syndrome or SDNS, and steroid resistant nephrotic syndrome), post-infectious glomerulonephritis (GN), thin basement membrane (TBM), and thrombotic microangiopathy (TMA), or a condition associated therewith.

Suitably, in the methods, composition and/or second medical uses of the presently disclosed compositions, a thiazolidinedione may be administered or formulated for administration at a dose of 0.01 mg or higher. Suitably, a thiazolidinedione is administered at a dose between 0.1-5000 mg/day.

Suitably, in the methods, composition and/or second medical uses of the presently disclosed compositions, a thiazolidinedione may be administered or formulated for administration in any suitable way, for example parenterally, enterally, or topically.

Suitably, in the methods, composition and/or second medical uses of the presently disclosed compositions, a thiazolidinedione may be administered or formulated for administration by oral, pulmonary, intravenous, intramuscular, or subcutaneous administration.

Another embodiment of the present disclosure includes use of a thiazolidinedione to decrease the cell damage or improve cell survival.

Another embodiment of the present disclosure includes use of a thiazolidinedione to reduce protein excretion in the urine.

One embodiment includes use of a thiazolidinedione to reduce the use of glucocorticoids for the treatment of nephrotic syndrome and other kidney diseases.

Another embodiment includes use of a thiazolidinedione to reduce the refractory nephrotic syndrome.

### EXAMPLES

The present invention is not to be limited in scope by the specific embodiments described herein. Indeed, various modifications of the invention in addition to those described herein will be apparent to those skilled in the art from the foregoing description and the accompanying Figures. Such modifications are intended to fall within the scope of the appended claims.

It is further to be understood that all values are approximate and are provided for description. All references cited and discussed in this specification are incorporated herein by reference in their entirety and to the same extent as if each reference was individually incorporated by reference.

### Example 1: Thiazolidinediones Improve Cell Viability

### Methodology

Cell Culture. The human podocyte cell line (CIHP-1) is a primary cell line isolated from human kidney. Podocytes are highly specialized, terminally differentiated cells with a complex cellular structure. They are a critical component of glomerular filtration. Cell cycle control, growth arrest and differentiation are key to the in vivo biology of podocytes. The conditionally immortalized podocyte cell line allows an in vitro process of maturation analogous to the development and maturation of podocytes in vivo. The result is a homogenous, stable cell source that shows expression of key antigenic markers of differentiated in vivo podocytes. These include the novel podocyte proteins nephrin, podocin, CD2AP and synaptopodin. This robust cell line is extensively characterized and validated.

The growth medium of CIHP-1 cells (Ximbio) was RPMI 1640 with 10% fetal bovine serum (FBS), 1-fold of ITS (insulin-transferrin-selenium), and 1-fold Pen/Strep. The culture condition was 33° C. with 5% CO₂. The cell detachment solution was Accutase® for 5 min at 33° C. The cell line passage ration was 1:3 every 3 or 4 days.

PAN-injury podocyte viability assay. Differentiated podocytes were either pretreated with dimethyl sulfoxide (DMSO) as vehicle, lobeglitazone, pioglitazone, rosiglitazone, or dexamethasone followed by puromycin aminonucleoside (PAN) treatment (5 μg/ml). Viability assays were performed after 3 or 5 days of treatments. Positive control cells (100% viable) did not receive any PAN treatment. For compound treatment, compounds were diluted to 20, 2, 0.2, 0.02 and 0.002 mM with DMSO. A volume of 1.8 μL of compounds was taken to 358.2 μL of culture medium and mixed. Media from plates were aspirated. A volume of 100 μL compound-added medium was added to each plate. Plates were placed in the incubator for 4 hours.

On day 0, CIHP-1 cells were seeded into two 96-well white plates using 5000 cell/well for 3 days (plate 1) or 2500 cell/well for 5 days (plate 2) treatment.

For compound treatment on Day 1, culture medium was aspirated. For each cell plate, 100 μl/well fresh medium with compounds was added for 4 hour. DMSO was used as control. After compound treatment for 4 hours, PAN was added to a final concentration of 5 μg/ml. No PAN treatment was used for the negative control. There was no medium change for the whole process.

On Day 4, for the first plate, Cell Titer-Glo (CTG) assay was used by adding 60 μl of CTG reagent into each well of the first plate, incubating at room temperature with shaking for 20 min, and then reading by the EnVision plate reader.

On Day 6, for the second plate, CTG assay was used by adding 60 μl of CTG reagent into each well of the second plate, incubating at RT with shaking for 20 min, and then reading by the EnVision plate reader.

### Results

### Lobeglitazone and Pioglitazone Prevent PAN-Induced Podocyte Injury

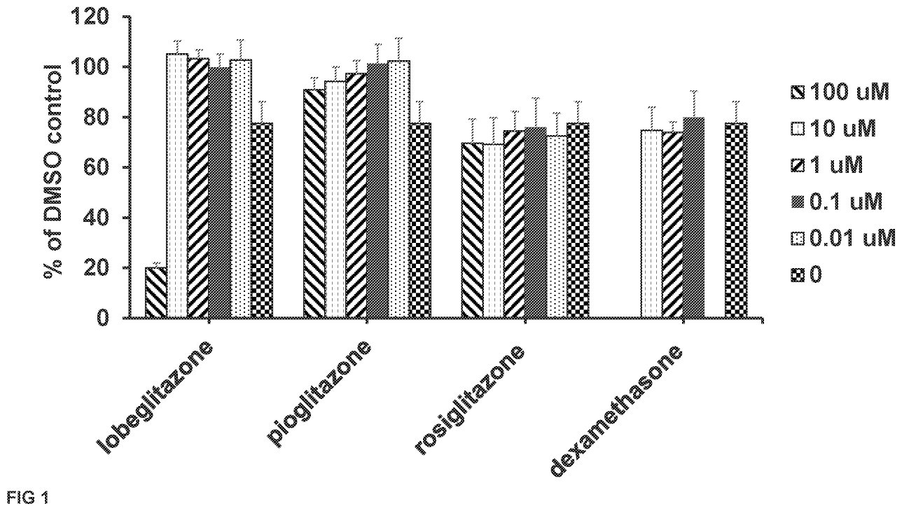

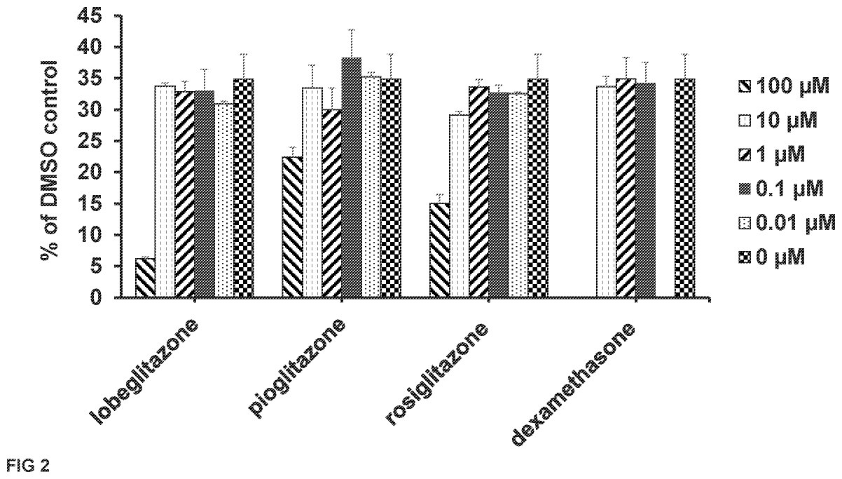

To test the direct protective effect of lobeglitazone, pioglitazone, rosiglitazone, and dexamethasone on cultured podocytes, cells were injured with 5 μg/ml PAN for 3 or 5 days, which resulted in decreased viability to 70 to 80% and 30 to 40%, respectively, compared with untreated cells (FIGS. 1 and 2).

Pretreatment of cells for 4 hours with lobeglitazone or pioglitazone significantly increased podocyte viability in a concentration- and time-dependent manner (FIG. 1). Specifically, pretreatment with lobeglitazone at 10 μM increased the viability to 105% after 3 days of PAN exposure, whereas simultaneous treatment with 0.01, 0.1, or 1 μM lobeglitazone increased the viability to 103% of that of the untreated control cells. Similarly, pioglitazone increased the viability to 91 to 94% with pretreatment at 10 μM. A similar viability result was observed post the treatment of pioglitazone at 0.01, 0.1, and 1 μM after 3 days of PAN exposure. A relatively lower viability in the pioglitazone-treated cells could be the result of drug toxicity to the podocyte cells, while the toxicity of lobeglitazone was only observed at 100 μM. While lobeglitazone and pioglitazone showed significant protection to PAN-injured podocytes, rosiglitazone and the glucocorticoid dexamethasone did not show any protection to PAN-injured podocytes at the concentrations used (0.01 to 100 μM, Table 1).

TABLE 1
Compounds effect on CIHP-1 proliferation
by CTG (PAN 3-day treatment)

| Compounds | | lobeglitazone | pioglitazone | rosiglitazone | dexamethasone |
|---|---|---|---|---|---|
| 100 | μM | 19.9% | 90.9% | 69.6% | |
| 10 | μM | 105.0% | 94.3% | 69.2% | 74.7% |
| 1 | μM | 103.3% | 97.3% | 74.5% | 73.9% |
| 0.1 | μM | 99.9% | 101.3% | 76.0% | 79.9% |
| 0.01 | μM | 102.7% | 102.4% | 72.4% | |

| | | | | |
|---|---|---|---|---|
| 0 | 77.5% | 77.5% | 77.5% | 77.5% |

values are shown as the percentage compared to the CIHP-1 proliferation with DMSO control (without PAN).

After 5 days of PAN injury, a less obvious change of viability was observed (FIG. 2). All drugs did not show significant improvement of podocyte viability after the treatment with lobeglitazone, pioglitazone, rosiglitazone, or dexamethasone at concentrations from 0.01 to 100 μM. More-frequent or more-sustained dosing of compounds will improve the viability of podocytes (Table 2).

TABLE 2
Compounds effect on CIHP-1 proliferation
by CTG (PAN 5-day treatment)

| Compounds | | lobeglitazone | pioglitazone | rosiglitazone | dexamethasone |
|---|---|---|---|---|---|
| 100 | μM | 6.2% | 22.4% | 15.0% | |
| 10 | μM | 33.7% | 33.4% | 29.1% | 33.6% |
| 1 | μM | 32.8% | 30.0% | 33.6% | 34.9% |
| 0.1 | μM | 33.0% | 38.3% | 32.7% | 34.2% |
| 0.01 | μM | 31.0% | 35.2% | 32.6% | |

| | | | | |
|---|---|---|---|---|
| 0 | 34.8% | 34.8% | 34.8% | 34.8% |

Note:
values are shown as the percentage compared to the CIHP-1 proliferation with DMSO control (without PAN).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of protecting kidney cells from damage and improving cell viability, comprising a step of contacting the cell with an effective amount of a thiazolidinedione selected from one or more of rosiglitazone, lobeglitazone, ciglitazone, darglitazone, englitazone, netoglitazone, rivoglitazone, troglitazone, and balaglitazone.

2. The method of claim 1, wherein the thiazolidinedione comprises lobeglitazone.

3. The method of claim 1, wherein the thiazolidinedione does not comprise rosiglitazone.

4. The method of claim 1, wherein the kidney cell is from a kidney tissue selected from renal cortex, renal medulla, renal papilla, renal pyramids, renal columns, fibrous capsule, hilum, renal artery, renal vein, renal pelvis, ureter, major calyx, and minor calyx.

5. The method of claim 1, wherein the kidney cell is a kidney glomerulus parietal cell, a kidney glomerulus podocyte, a kidney proximal tubule brush border cell, a loop of Henle thin segment cell, a thick ascending limb cell, a kidney distal tubule cell, a collecting duct principal cell, a collecting duct intercalated cell, or an interstitial kidney cell.

6. The method of claim 1, wherein the cell is an animal cell.

7. The method of claim 7, wherein the cell in a human cell.

8. The method of claim 1, wherein the cell is treated in vitro.

9. The method of claim 1, wherein the cell is treated ex vivo.

10. The method of claim 1, wherein the cell is treated in vivo.

11. The method of claim 1, wherein the cell is in a subject having a disease or disorder or is at risk of having the disease or disorder.

12. The method of claim 11, wherein the disease or disorder is selected from one or more of age-associated glomerulonephropathy, AL amyloidosis, Alport syndrome, amyloidosis (amyloid nephropathy), ANCA vasculitis, anti-GBM disease (Goodpasture syndrome), C1q nephropathy, C3 glomerulopathy, collapsing glomerulopathy, collapsing glomerulonephropathy, congenital nephrotic syndrome of the Finnish type (CNSF), cryoglobulinemia, diabetes, Denys-Drash Syndrome, diabetic glomerulonephropathy, diabetic nephropathy, diffuse mesangial sclerosis (DMS), Fabry disease, fibrillary glomerulonephritis (GN), focal segmental glomerulosclerosis (FSGS), heavy chain deposition disease, hypertensive nephropathy, IgA vasculitis, IgA nephropathy, IgM nephropathy, immune and inflammatory glomerulonephropathy, immunotactoid glomerulopathy, light chain deposition disease, lupus, lupus nephritis, membranous nephropathy, membranoproliferative glomerulonephritis (MPGN), mesangial proliferation, mesangial sclerosis, myeloma kidney, minimal change disease, nephrotic syndrome, post-infectious glomerulonephritis (GN), thin basement membrane (TBM), thrombotic microangiopathy (TMA), and a condition associated therewith.

13. The method of claim 12, wherein the nephrotic syndrome is frequent relapsing nephrotic syndrome, steroid dependent nephrotic syndrome, or steroid resistant nephrotic syndrome.

14.-33. (canceled)

34. A method of treating nephrotic syndrome in a mammal comprising administering an effective amount of a thiazolidinedione selected from one or more of rosiglitazone, lobeglitazone, ciglitazone, darglitazone, englitazone, netoglitazone, rivoglitazone, troglitazone, and balaglitazone to the mammal.

35. The method of claim 34, wherein the nephrotic syndrome is frequent relapsing nephrotic syndrome (FRNS) or steroid dependent nephrotic syndrome (SDNS).

36. A method of treating glomerular disease in a mammal comprising administering an effective amount of a thiazolidinedione selected from one or more of rosiglitazone, lobeglitazone, ciglitazone, darglitazone, englitazone, netoglitazone, rivoglitazone, troglitazone, and balaglitazone to the mammal.

37. The method of claim 36, wherein the glomerular disease is focal segmental glomerulosclerosis (FSGS), minimal change disease, or membranous nephropathy.

38. The method of claim 34, wherein the thiazolidinedione comprises lobeglitazone.

39. The method of claim 34, wherein the thiazolidinedione does not comprise rosiglitazone.

40. The method of claim 36, wherein the thiazolidinedione comprises lobeglitazone.
